(19) United States
(12) Patent Application Publication
(10) Pub. No.: US 2005/0281853 A1
(43) Pub. Date: Dec. 22, 2005

(54) Skin compatible cosmetic compositions and delivery methods therefor

(76) Inventors: Charles A. Fox, Fair Lawn, NJ (US); Terrence S. McGrath, Boca Raton, FL (US)

Correspondence Address: EDWARDS & ANGELL, LLP, P.O. BOX 55874, BOSTON, MA, 02205, US

(21) Appl. No.: 11/212,521

(22) Filed: Aug. 26, 2005

Related U.S. Application Data
(63) Continuation-in-part of application No. 10/366,845, filed on Feb. 14, 2003.
(60) Provisional application No. 60/357,466, filed on Feb. 15, 2002.

(51) Int. Cl. A61K 31/728; A61K 31/19; A61K 7/00

(52) U.S. Cl. 424/401; 514/54; 514/557

(57) ABSTRACT

A unique delivery method for providing optimal pH in topical skin formulations is described. Cosmetically acceptable skin formulations prepared with selected weak acids neutralized with ammonia will gradually increase in acidity after topical application. The initial neutral pH changes to a pH compatible with normal skin processes important for skin lipid barrier renewal and sloughing of cells from the stratum corneum.

### CROSS REFERENCE TO RELATED APPLICATIONS

This application is a continuation-in-part of U.S. patent application Ser. No. 10/366,845 filed Feb. 14, 2003, which takes benefit from U.S. provisional application Ser. No. 60/357,466, filed Feb. 15, 2002, herein incorporated by reference in the entirety.

### BACKGROUND OF THE INVENTION

1. Field of the Invention

The invention relates to topical skin compositions, in particular to skin compatible cosmetic formulations and systems including cosmetic formulations that effectively deliver skin-enhancing ingredients to the skin surface.

2. Description of Background Art

The skin acts as a protective barrier against internal damage, which may arise from exposure to chemicals and infectious microorganisms as well as from physical damage. Healthy skin protects against environmental damage that may arise from contact with free radicals found in pollutants and from ultraviolet radiation. As the largest single organ in the body, the skin has a multiple layer structure, each serving separate vital metabolic and protective functions.

Skin is made up of three distinct layers; the epidermis, which is a thin, translucent outer layer; the dermis, which is a thicker inner layer; and a subcutaneous layer of fat. There is a thin membrane below the epidermis, the so-called basement membrane, which attaches to the dermis, allowing nutrients and oxygen to move up from the deeper layers of the skin to the epidermal layer. A subcutaneous fat layer made up of fat-filled cells known as adipose cells lies below the dermis. This fat layer contains larger blood vessels and nerves.

The internal surface of the epidermis serves as an incubator for cell multiplication. New basal cells, which originate from the surface of the basal membrane, continuously push older cells epidermal cells toward the skin surface causing them to flatten and overlap to form a tough layer of dead skin that gradually sloughs. The top of the epidermis, or stratum corneum (SC) is a cornified layer of tightly packed flattened cells, which act as a outer skin covering resistant to physical damage and to prevent water loss from underlying layers. The constant renewal of the epidermal surface by this process plays an important role in preventing moisture loss from the body and maintaining homeostasis.

The lipids in the epidermis assist normal skin in regulating natural water loss in the SC. The epidermis also has several hydrolase class enzymes, which are important in the formation of the lipids that make up the protective barrier layer.

The dermis, unlike the epidermis, performs a variety of functions, due to the presence of nerve endings, blood vessels, melanin and sebaceous cells. It is a relatively thick layer that can be up to 3 millimeters thick. The dermis is composed largely of bundles of collagen, which run horizontally through the dermis and are imbedded in a jelly-like material called ground substance. The ground substance contains hyaluronic acid, as well as glycoproteins, which are able to attract and hold large amounts of water. Collagen is up to 75% of the weight of the dermis and contributes to skin elasticity and resilience of the skin. Elastin is also found in the dermis, contributing about 5% of the weight.

Numerous projections or papillae extend from the surface of the dermis, which fit into depressions on the inner surface of the epidermis so that the two layers are securely locked together. The dermis also lies directly above the subcutaneous fat layer to which it connects with finger-like structures known as ret pegs. The fat layer is loosely attached to muscle and bone by connective tissue so that the skin does not remain rigidly in place when muscle movement occurs.

Because human skin performs several functions, including preventing excessive loss of body moisture and surface lubrication from the internal oil glands, the metabolic activity of the skin is an important factor to consider in the development of any skin care product that will be in contact with the skin, particularly in cosmetic preparations. Several factors need to be considered, including maintaining and enhancing moisture retention and safety, because many of these products remain in contact with the skin for several hours. Therefore considerable attention has been paid to enhancing moisturization in order to prevent the skin from becoming dry and to maintain the water level of the SC, which is about 15% under normal conditions. When water is less than about 10%, the ability of the SC to retain body moisture, and to perform its protective function, is seriously reduced.

Normal human skin requires about 30 days for a cell produced by the basal layer to move through the epidermis to the surface; however, this process is accelerated when the SC cells are lost more quickly than normal, such as by physical injury or free radical damage. The lipids in the epidermis are important in helping to regulate natural water loss, which naturally occurs through the skin by a process of transeipdermal water loss (TEWL). The basal layer is composed of 70-75% water, but the SC is only about 10-15%, so that if the skin becomes dry, normal TEWL is disrupted, making proper hydration of the SC particularly important. Normally the epidermis is about 35 microns thick when dry but when fully hydrated may be up to 48 microns thick.

Epidermal permeability barrier homeostasis depends on post secretory processing of polar lipid precursors into nonpolar products within the stratum corneum (SC) interstices by hydrolase enzymes.

Normal pH of the skin epidermis is about pH 5.0, which appears to be optimal for maintaining the lipid barrier. At higher pHs, around neutral or basic, the lipid barrier deteriorates because the repair process is inhibited (Mauro, 1998). Epidermal permeability barrier homeostasis depends on post secretory processing of polar lipid precursors into nonpolar lipid products within the stratum corneum (SC) interstices by hydrolase enzymes. Beta-glucocerebrosidase, for example, is an important SC hydrolase, which exhibits optimal activity at pH 5.5.

The epidermal surface has been known for many years to be acidic (Mauro, et al., 1988), but until recently the importance of the SC pH in barrier homeostasis has remained largely undefined. Studies by Loden and Maibach (2000) showed that an acidic pH is conducive to barrier recovery when skin is damaged by exposure to acetone, an irritating and drying organic liquid commonly used as a solvent. At neutral or basic pH, barrier recovery was significantly delayed, indicating that an extracellular pH in the SC is required for normal extracellular lipid processing and normal barrier homeostasis. FIG. 1 shows the natural pH repair zone for the skin.

Most creams and lotions on the market are neutral or slightly alkaline and are relatively stable toward pH changes after topical application. Unfortunately, neutral or alkaline pH is not favorable for repair of the skin lipid barrier so that while moisturization may be provided by the formulations, the natural and ongoing process of moisture evaporation from the skin is impaired. Acidic cosmetic products such as retinoic acid are available, often by prescription, but they are often irritating to the skin. Most facial over-the-counter products do not provide the optimal pH for the hydrolase activity important in lipid processing for SC barrier repair.

Shelf life and stability are important for successful commercialization of cosmetic products, which may remain in retail stores or in the consumer home for long periods of time before use. Most base cosmetic formulations are prepared as emulsions, either water in oil or oil in water, and are generally formulated at pH 6.5 to 8.0 in order to maintain stability and a shelf life up to at least two years. As such, the formulations are stable even after topical skin administration but because these compositions remain at or near neutral pH after application, they are not compatible with the optimal acidic pH required in the SC for normal extracellular lipid processing and normal barrier homeostasis.

Emulsifiers are used in virtually every cosmetic cream, liquid or lotion in order to prevent separation of the different ingredients conventionally used in facial care cosmetics. Many of the active agents used in facial care cosmetics are highly lipophilic in nature and are insoluble in aqueous media or only partially soluble in alcohol. Unfortunately, alcohol is drying to the skin and one purpose of most facial cosmetics is to maintain and/or enhance skin hydration. These preparations are typically formulated with 5-7% emulsifying agent at a pH of 6.5-8.0 to insure product stability. Most also contain an average of about 75% water. After application, the water evaporates leaving a residue of emulsifier on the skin. This residue can interact with natural skin lipids, causing a loss of protective barrier lipids and a drying effect on the skin. Depending on the nature of the emulsifier, there may also be a certain amount of damage to the skin; however, at the near neutral pH of the topical product, repair processes in the SC are inhibited because optimal pH for proteases involved in desquamation of horny cells is about 4 to 5.

Low acidity, below 4.0, will often cause burning or irritation. Products having a pH of about 6 have good shelf life, but do not promote rebuilding of the skin's protective barrier, simply because the enzymes required for the rebuilding process have low activity at pHs near neutral and only function well at a lower pH.

### DEFICIENCIES IN THE ART

Many cosmetic and cosmeceutical products currently marketed as skin products are formulated to ensure stability and maintain an acceptable product appearance. In order to incorporate effective moisturizing agents in such products, use of emulsifiers is usually necessary in oil/water formulations. In many instances especially when salts of fatty acids are used as the emulsifying agent, a pH adjustment is necessary to form a stable emulsion. While lower pH is recognized as desirable for maintaining proper lipid barrier homeostasis, low pH facial products have not effectively enhanced skin hydration or have caused skin irritation due to low pH.

Accordingly, there is a need for safe and effective facial hydrating cosmetic formulations that are stable under storage conditions yet provide an optimal acidic pH after application in order to maintain normal activity of the SC enzymes important in epidermal permeability barrier homeostasis.

### SUMMARY OF THE INVENTION

The present invention concerns methods for preparing topical skin formulations that overcome two important aspects of cosmetic preparations; stabilized compositions with good shelf life; and a delivery system that provides an optimal pH environment for normal skin moisturizing and regeneration after topical administration.

Skin moisturizers, particularly those intended for facial application, are conventionally prepared in cosmetically acceptable base formulations, well known and practiced by those skilled in the art. Such formulations typically include combinations of ingredients selected from emulsifiers, emollients, preservatives, skin conditioners, humectants, as well as fragrance, opacifying agents, vitamins, antioxidants, sunscreen agents and the like. Because formulation stability is important, pH is adjusted to about neutral; i.e., 6.0-7.0. This pH is particularly advantageous in preparing a stable suspension comprising mixtures of lipophilic and lipophobic ingredients and in enhancing shelf life of the product.

Adjustment of the pH in most cosmetic formulations is typically accomplished with a strong base, such as triethanolamine, sodium hydroxide or potassium hydroxide, to bring the pH to neutral or close to a neutral pH. The strong base is effective in increasing the pH, which may be initially acidic if fatty acids or weak organic acids are part of the formulations. When the formulations are applied to the skin, there is little change in pH, so that the pH at the skin surface, normally somewhat acidic, is exposed to a neutral or slightly alkaline pH environment.

In contrast to typical processes for adjusting pH of a cosmetic base, the invention in one aspect is the use of ammonium hydroxide to adjust initial pH to a near neutral value. The base formulation is stabilized at this pH, comparable to what is achieved using a strong base. Any weak acids included in the base formulation are converted to their ammonium salts during the pH adjustment process. After formulation, but prior to use, the formulations remain at or near neutral pH and consequently exhibit stability and good shelf life.

A surprising effect of adjusting pH in cosmetic formulations with ammonium hydroxide is that upon application to the skin, the acidity of the formulation slowly increases until it reaches a value determined by the pKa of the weak acids comprising the ammonium salts of weak acids used in the preparation. By selecting any of a number of weak organic acids that have a pKa around 4.5 to 5.5 to include in the base formulations, it is possible to ensure that the pH of the preparations when applied to the skin will adjust to a pH that is near the normal pH of the SC of healthy skin. This means that skin renewal and repair processes will not be impaired by an increase in pH due to prolonged contact with formulations that are neutral or alkaline.

There are a number of weak organic acids that can be used in preparing cosmetic formulations. Typical weak acids added in various amounts to currently marketed formulations include glycolic acid, isosteric acid, stearic acid, palmitic acid, behenic acid, cerotic acid, and lactic acid. Some of these acids are particularly desirable in the practice of the present invention because in addition to being weak acids, they generally have humectant or skin conditioning properties. For the purposes of the present invention, it may be desirable to add a greater amount or an additional weak organic acid in order to attain a lower pH than would be achieved if, for example, only small amounts, or none, of only one weak organic acid were otherwise present in the formulation. Weak acids for consideration include citric, tartaric, ascorbic, acetic acid, and malic acid.

Accordingly, an aspect of the invention is a pH neutral cosmetically acceptable formulation that gradually adjusts to an acidic pH when in contact with the skin. A typical topical composition, comprises an ammonium salt of a weak organic acid in a cosmetically acceptable base formulation such that the composition releases ammonia when in skin contact to provide a gradual increase in acidity to a pH controlled by the pKa of the organic weak acid and the ammonium salt of the acid.

The pH of the base formulation as prepared is near neutral pH, typically between 6.5 and 7.0, and is unchanged until applied to the skin. After application, the acidity of the composition gradually increases to pH 4.0-4.5, generally over a period of one to four hours after application to the skin. The time required to reach a constant acidity, however, may vary and will depend not only on the weak acids included in the compositions but also on the external temperature and the amount of water on the skin surface and provided in the formulation.

As practiced in the art, other ingredients may be added to the base formulation, such as vitamins, waxes, fatty acids, antioxidants and humectants. Popular additions include tocopheryl acetate, vitamin A palmitate, aloe vera gel, beeswax, sodium hyaluronate, cholesterol, lanolin, glycerin, fatty acid esters, vegetable oils, waxes and fragrances.

Additional optional ingredients include grape seed extract, idebenone, centella Asiatic extract, DMAE, ethoxydiglycol, arnica, ginko biloba, ginseng and various forms of retinoic acid such as retinyl A and retinyl palmitate. Some of these compounds have therapeutic action; for example, centella Asiatic extract is believed to act as a vasoprotectant; DMAE is touted as having anti-wrinkle properties; arnica is a herb said to reduce bruising; and idebenone has antioxidant properties. There are numerous botanicals and chemicals that have been added to cosmetic formulations. Whether or not some of them have a beneficial effect may be open to question and is likely to depend on other ingredients in the formulation and on the amount present.

Selection of the weak organic acid can be on the basis of the pH desired, which will generally be in the pH 5 range. This is the optimal pH for activity of beta-glucocerebrosidase and other enzymes essential to maintaining impermeability of the lipid barrier beneath the SC.

What determines the final pH of the disclosed compositions is a function of the presence of several different species in addition to the water content of the preparation. For most facial preparations, the water content is fairly high, on the order of 60-70%. Final pH is determined by the relative amounts of ionized ammonium salt, the free acid, environmental factors and loss of ammonia. The pKa of a selected acid does not necessarily predict the ultimate pH in these formulations after application; however, ionization constants, from which pKa can be calculated, may provide guidance for selection. The pH must be tested for different formulations to determine the final pH.

While most facial preparations sold as day or overnight moisturizers preferably incorporate weak acids such as stearic, some preparations are formulated to increase sloughing of dead cells, thereby stimulating cell turnover, by incorporating alpha or beta hydroxy acids, glycolic acid, citric acid, salicylic acid or the like. These acids tend to provide a final more acidic pH, near 4.0, depending on the other ingredients in the composition, but the resulting pH should not significantly inhibit normal activity of lipid barrier hydrolases and other enzymes.

Yet another aspect of the invention is the formulation of relatively stronger weak acids into conventional cosmetic moisturizing treatments for use as sloughing agents or, when salicylic acid is present as an active ingredient, as an acne treatment. In such cases, the ideal skin rejuvenating pH may not be achieved except as the pH gradually changes from the initial pH near neutral; however, the advantage may be a gradual change to an acidic environment with the pH at least passing through a pH that may be optimal for skin repair enzymes.

A further aspect of the invention is a topical skin delivery system. The system is comprised of a base formulation prepared from a weak organic acid, a moisturizer in a cosmetically acceptable base and any of a number of optional ingredients. The pH is adjusted to a near neutral value, around pH 6.5 to 7.0, with ammonium hydroxide (aqueous ammonia). Once a stable formulation is prepared, the pH remains near neutral until applied to the skin. Skin moisture causes a gradual increase in acidity as the ammonium salt of the weak acid dissociates and the acidity approaches a value determined by the pKa of the acid as well as any ions or salts of weak acids that may be present. The delivery system in effect is initiated by skin contact and is due to the particular nature of equilibria involving ammonium salts of weak acids.

A skin delivery system may also be engineered to directly deliver a cosmetic topical preparation using an application designed to premix separate components prior to application. Accordingly, such a delivery system for providing an on-demand topical skin moisturizing preparation will include a means for releasing aliquots of a stabilized pH neutral cosmetic facial composition and a separately compartmentalized cosmetically acceptable acidic composition such that release of the aliquots results in an acidic mixture directly applicable to the skin. This modification effectively delivers a cream, lotion, gel or liquid directly to the skin at a selected acidic pH. This may be particularly advantageous for acne treatments or where skin sloughing is desired.

An exemplary delivery device is shown in FIG. 2. A and B are separate chambers, each containing respectively a cosmetic base formulation and an acidic composition. The base may be a gel, cream or liquid and normally will be a syringeable cream stabilized around neutral pH. The acidic composition may be formulated from any of several components, but is preferably buffered so as to provide a pH of 4.5-5.5 when mixed with the cosmetic base. C represents plungers or other means for pushing the A and B components down through a one-way pressure movable diaphragm D to mix in the mixing chamber M. The mixture may be immediately deposited on the skin, or optional means engineered to allow additional time for mixing before application.

In such a two-part delivery system, it will be important to select skin compatible formulations for each component. The acidic component will mix with a stable neutral base cosmetic formulation so that the pH adjusts to a predetermined value, generally selected to be in the range of 5.0-5.5. The proper choice of acidic material will ensure a rapid pH change in the mixture, preferably within a few minutes. Some attention will have to be paid to the type of acid used and its interaction with the base cosmetic formulation, which preferably will include an emulsifier. The emulsifier will accomplish ensure a reasonably homogeneous mixing of the components and can be selected to enhance moisturizing properties.

Definitions

As used herein, cosmeceuticals is a hybrid term that incorporates the concept of improving skin appearance by a combination of active ingredients, one or more of which may have a therapeutic effect.

Cosmetic formulations refer to compositions that contain ingredients that typically improve the appearance of the skin but generally do not have a significant therapeutic effect.

Pharmaceutical formulations refer to compositions that contain one or more active ingredients that may have a therapeutic effect, such as an anti-inflammatory or anti-microbial effect. The type or amount of active ingredient may be regulated under Federal Drug Administration guidelines.

Weak organic acids are carbon-based acids that do not completely dissociate in aqueous media, generally having a pH no lower than about 3.

Emulsifiers are agents that promote stabilized emulsions of lipid and non-lipid ingredients. Generally there are four types of emulsifiers; anionic, cationic, nonionic and quaternary.
pH is by definition the negative log of the hydrogen ion concentration in solution; i.e.,

$$pH = pKa + log \frac{[ A - ]}{[ HA ]}$$

as set forth in the Henderson-Hasselbach equation defining the relationship between pH and pKa. [A−] is the molar concentration of the salt (dissociated species) and HA is the concentration of the undissociated acid. When the concentrations of the salt and acid are equal, the pH of the system is equal to the pKa of the acid. The pKa is a dissociation constant determined by the strength of an acid or base. As used herein, pH refers to a measured pH value and not to a calculated value requiring what may be complex determination of activities and measurement of very low concentrations.

Oil in water emulsions consist of tiny drops of oil in an aqueous base, which is the continuous phase. Alternatively, water in oil emulsions consist of a water phase dispersed in an oil. In either type, the dispersed phase is finely divided within the emulsion.

Nanoemulsions are emulsions comprised of extremely tiny particles or drops. In some formulations, this may provide certain advantages in enhanced skin absorption of certain ingredients and/or in providing more stable formulations.

The terms “formulations”, “preparations”, “compositions” and the like are not intended to have significantly different meanings; rather, they are intended as general descriptive terms for mixtures of ingredients.

### BRIEF DESCRIPTION OF THE DRAWINGS

FIG. 1. Shows the natural pH repair zone for skin.

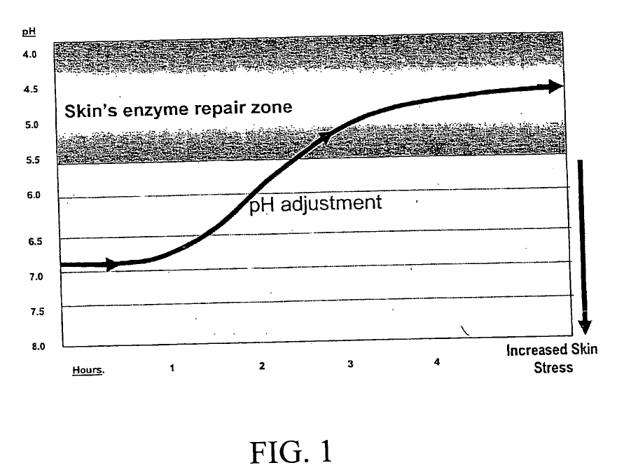

FIG. 2. Represents a device for premixing creams or liquids for application to the skin.

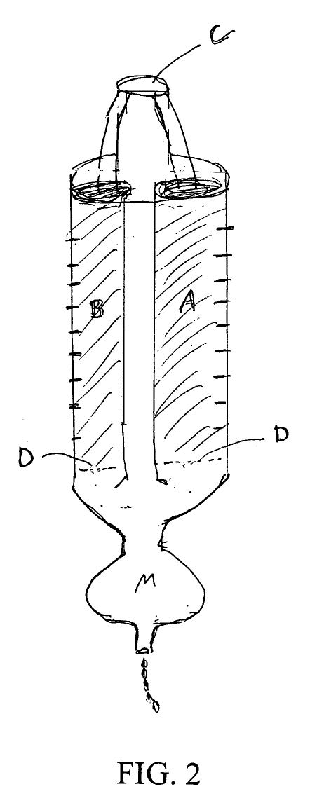

### DETAILED DESCRIPTION OF ILLUSTRATIVE EMBODIMENTS

Historically, the acidity of the epidermal surface has been long recognized but why it was important was best elucidated by Klingman (2000) when studies were conducted on permeability barrier recovery by measuring changes in transepidermal water loss after exposing normal skin to acetone, a low weight, volatile organic ketone that acts both as a drying agent and a skin irritant. Changes in transepidermal water loss were measured using ruthenium tetroxide (RuO4) postfixation and assessing beta-glucocerebrosidase activity by in situ zymography at neutral and acidic pH. Barrier recovery proceeded normally when acetone treated skin was exposed to solutions buffered to an acidic pH, but was slower when the treated skin was exposed to neutral or alkaline pH.

Further studies have showed that addition of Ca²⁺ and K⁺ in alkaline buffers results in even slower barrier recovery than with an alkaline buffer alone. At pH 7.4, but not pH 5.5, there is a persistence of immature, extracellular lamellar membrane structures and a marked decrease in the in situ activity of beta-glucoerebrosidase. The pH-dependent alteration in barrier recovery appears to occur through a mechanism that was independent of Ca²⁺ or K⁺ controlled lamellar body secretion since both the formation and secretion of lamellar bodies proceeded comparably at pH 5.5 and 7.4.

The results of this study suggested that an acidic extracellular pH would be necessary for the initiation of barrier recovery and that any significantly effective moisturizing facial formulation would have to provide an ideal pH for barrier recovery if the desired effects were to be provided and normal skin processes not to be inhibited.

The present invention addresses some of the disadvantages in using facial moisturizers that do not provide an environment supportive or conducive to barrier recovery. Compositions for use as cosmetics, which adjust from higher to lower pH after topical application, have been developed. The compositions are formulated to provide a pH of about 4.5 by incorporating a weak organic acid as an important component. The pKa of the acid determines the final pH of the composition after application to the skin and use of selected organic acids assures that the acidity of the composition will not cause damage to normal skin. The inventive compositions can remain safely in contact with the skin over relatively long periods of time because the acidity can be controlled by selection of the appropriate weak acids and use in the form of their ammonium salts.

Cosmetic formulations are somewhat of an art and must have active ingredients combined in a base composition that is stable, resistant to microbial contamination, effective, safe, cost effective to produce and attractive to purchasers. In general, most cosmetic moisturizing products are water-based emulsions of skin conditioning agents, humectants, emollients, preservatives, dispersants and emulsifiers. There are many optional ingredients, which may or may not have an effect on skin chemistry; these include vitamins, sunscreens, fragrance, antioxidants, botanicals, color and opacifying agents, among others. Nevertheless, in order to obtain homogeneous mixtures of these various ingredients, varying amounts of emulsifying agents are typically required.

Emulsification is particularly important in the cosmetic industry because the formulations are generally in the form of creams or lotions formed from a mixture of lipophilic and hydrophilic ingredients. Because oil and water do not mix well, one or more emulsifiers may be used in order to achieve homogeneous and stable preparation.

Cosmetically acceptable emulsifiers are well-known and, as indicated, are extensively used in skin product formulations. Triethanolamine stearate, potassium stearate, and diethanolamine cetyl phosphate are examples of anionic emulsifying agents; cetyl alcohol and ceteth-20, glycerylstearate (and) PEG-100 stearate are examples of nonionic emulsifying agents; and steapyrium chloride and distearyldimoniuim chloride are typical cationic emulsifying agents. Amphoteric emulsifiers, as the name suggests, can act either as cations or anions, depending on the polarity or charges of the different species in the formulation.

The list of emulsifiers is extensive and is not necessarily limited to chemical species whose only function is emulsification, since some compounds may have this property in addition to skin softening properties. For example, polyethylene glycols (PEG) are commonly attached to laurate, beeswax, glycerides, stearates, oleates, lanolin, oils and fatty acids, the variations of which give rise to a large number of choices of materials useful as emulsifying agents.

Glycerin is the most commonly used humectant, while other popular emollients include isohexadecane, isopropyl isostearate, mineral oil, silicones, low and high molecular weight fatty acids, C13-14 isoparaffin, tocopherol acetate, and dimethiconol. Commonly used emulsifiers are sorbitan fatty acid esters such as sorbitan stearate, glyceryl stearate, stearic acid, cetyl alcohol, polyethylene glycol esters, ethers of fatty acids, fatty alcohols such as Ceteth-20, polysorbate 20, PEG-100 stearate and the like. Methyl paraben, ethyl paraben, propyl paraben and benzyl alcohol are commonly used preservatives. Particular moisturizers found in some products include hyaluronic acid and ceramides.

The combination of ingredients in cosmetic products rarely forms a homogeneous preparation without an emulsifier. Mixing procedures can contribute to an effective emulsification process but most commercially successful products contain water and at least a small percentage of lipophilic agents, which are relatively unstable unless an emulsifier is used to inhibit separation of the lipophilic components.

### EXAMPLES

### Example 1

### Skin Moisturizing and Sloughing Preparation

In this example, glycolic acid ultimately is the prime determinant in the pH level reached. As shown in examples 2 and 3, other acids can be used and still provide a skin sloughing action.

| Ingredient | Percent (w/w) |
|---|---|
| Part 1 | |
| Stearic Acid | 2.55 |
| Cetyl Alcohol | 1.70 |
| Glyceryl Stearate | 1.25 |
| Isopropyl Stearate | 0.42 |
| Caprylic/Capric Triglyceride | 0.25 |
| Lanolin | 0.42 |
| Part 2 | |
| Deionized Water | qs 100.00 |
| Glycolic Acid (70%) | 7.00 |
| Ammonium Hydroxide (28%) | qs to pH 6.7 |
| Part 3 | |
| Propylene Glycol | 7.50 |
| Steareth-20 | 0.02 |
| Part 4 | |
| Germaben II (ISP) | 1.00 |
| Part 5 | |
| Fragrance | 0.10 |

### Example 2

### Skin Facial Moisturizing and Sunscreen Preparation

| Ingredient | Percent (w/w) |
|---|---|
| Part 1 | |
| Octyl Methoxy Cinnamate | 7.00 |
| Benzophenone-3 | 3.00 |
| Avobenzone | 2.00 |
| Stearic Acid | 2.30 |
| Cetyl Alcohol | 1.50 |
| Glyceryl Stearate | 1.50 |
| Dimethicone | 1.00 |
| Tocopheryl Acetate Cosmetic Grade | 0.50 |
| C₁₂₋₁₅ Alkyl Benzoate | 0.35 |
| Stearoxytrimethylsilane | 0.25 |
| Part 2 | |
| Vitam A Palmitate | 0.10 |
| Tetrahexadecyl Ascorbate | 0.10 |
| Part 3 | |
| Deionized Water | qs 100.00 |
| Lactic Acid (88%) | 5.00 |
| Disodium Edetate | 0.20 |
| Part 4 | |
| Deionized Water | 9.80 |
| Propylene Glycol | 7.50 |
| Aloe Vera Gel (200X) | 0.10 |
| Steareth-20 | 0.02 |
| Part 5 | |
| Ammonia Solution Strong (27%) (26° Baume) | qs to pH 6.7-6.9 |

### Example 3

### Acne Moisturizing and Treatment Cream

| Ingredient | Percent (w/w) |
|---|---|
| Part 1 | |
| Caprylic/Capric Triglyceride | 5.00 |
| Glyceryl Stearate | 3.00 |
| Salicylic Acid | 2.00 |
| Cetyl Alcohol | 1.50 |
| Stearic Acid | 1.30 |
| Beeswax | 1.00 |
| Cholesterol | 0.25 |
| BHT | 0.05 |
| Part 2 | |
| Deionized Water | 47.17 |
| Propylene Glycol | 5.00 |
| Sodium Hyaluronate | 3.00 |
| Sodium PCA | 2.00 |
| Saccharide Isomerate | 1.00 |
| Disodium Edetate | 0.20 |
| Part 3 | |
| Deionized Water | 9.80 |
| Propylene Glycol | 7.50 |
| Aloe Vera Gel (200X) | 0.10 |
| Steareth-20 | 0.02 |
| Part 4 | |
| Ammonia Solution Strong (27%) (26° Baume) | qs to pH 6.7-6.9 |

### Example 4

### Acne Spot Treatment Gelee

| Ingredient | Percent (w/w) |
|---|---|
| Part 1 | |
| Deionized Water | qs 100.00 |
| Natrosol 250 HR | 2.00 |
| Part 2 | |
| Propylene Glycol | 7.00 |
| Salicylic Acid | 2.00 |
| Part 3 | |
| Ammonia Solution Strong (28%) (adjust to pH 6.0-6.9) | 0.90 |
| Part 4 | |
| Aloe Vera Extract | 0.10 |
| Chamomile Extract | 0.10 |
| Actiphyte of Comfrey BG-50 (Active Organics) | 0.10 |
| Actiphyte of Gingko BG-50 (Active Organics) | 0.10 |
| Actiphyte of Green Tea BG-50 (Active Organics) | 0.10 |
| Actiphyte of Witch Hazel BG-50 (Active Organics) | 0.10 |
| Actiphyte of Yarrow BG-50 (Active Organics) | 0.10 |
| Part 5 | |
| Germaben II | 1.00 |
| Part 6 | |
| Fragrance | 0.03 |
| Polysorbate 20 | 1.00 |

### Example 5

### Line and Wrinkle Reducing Lotion

| | Percent (w/w) |
|---|---|
| Part 1 | |
| Stearic Acid | 2.55 |
| Cetyl Alcohol | 1.70 |
| Glyceryl Stearate | 1.25 |
| Dimethicone | 1.00 |
| Caprylic/Capric Triglyceride | 0.75 |
| Cholesterol | 0.10 |
| Lecithin | 0.10 |
| Isocetyl Alcohol (and) Ceramide 3 | 0.01 |
| Part 2 | |
| Tocopheryl Acetate | 1.00 |
| Tetrahexadecyl Ascorbate | 1.00 |
| Vitamin A Palmitate | 0.10 |
| Part 3 | |
| Deionized Water | qs 100.00 |
| Matryxyl | |
| Disodium EDTA | 0.20 |
| Aloe Vera Powder | 0.10 |
| Centella Asiatica Extract | 0.10 |
| Green Tea Extract | 0.10 |
| Grape Seed Extract | 0.10 |
| Palmitoyl Hexapeptide-3 | 6.00 |
| Part 4 | |
| Ammonium Hydroxide Strong (28%) (qs to pH 6.5-7.0) | 0.25 |
| Part 5 | |
| Propylene Glycol | 7.50 |
| Part 6 | |
| Germaben II | 1.00 |

### Example 6

### pH of Several Popular Cosmetic Formulations

Table 1 is a comparison of the formulations disclosed herein with several popular facial preparations. None of the commercial products tested changed significantly from the initial pH, while formulations prepared with ammonium hydroxide as the neutralizing agent at initially the same neutral pH, became more acidic over a period of 8 hours, at which point the pH was 4.5. The lowest pH of the other products tested was 6.5.

TABLE 1
pH*

| Product | Initial | 1 hr | 2 hr | 3 hr | 8 hr |
|---|---|---|---|---|---|
| Vaseline ® Intensive Care | 7.5 | 7.5 | 7.5 | 7.5 | 7.5 |
| Advanced Healing Lotion | | | | | |
| (Lot 04219PP10) | | | | | |
| Vaseline ® Intensive Care | 7.0 | 7.0 | 7.0 | 7.0 | 7.0 |
| Dry Skin Lotion | | | | | |
| (Lot 67999L) | | | | | |
| Lubriderm ® Skin Therapy | 7.5 | 7.5 | 7.5 | 7.5 | 7.5 |
| (Lot 67999L) | | | | | |
| Neutrogena ® Moisture for | 7.5 | 7.5 | 7.5 | 7.5 | 7.5 |
| Sensitive Skin (Lot 1L9) | | | | | |
| Ponds ® Nourishing | 6.5 | 6.5 | 6.5 | 6.5 | 6.5 |
| Moisturizer Lotion | | | | | |
| (Lot 01190H02) | | | | | |
| Neutrogena ® Intensified | 7.0 | 7.0 | 7.0 | 7.0 | 7.0 |
| Day Moisture Lot 1M9) | | | | | |
| L'Oreal ® Active Daily | 6.0 | 6.0 | 6.0 | 6.0 | 6.0 |
| Moisture Lotion | | | | | |
| (Lot PV108) | | | | | |
| Nivea ® Visage | 7.0 | 6.5 | 6.5 | 6.5 | 6.5 |
| (Lot 99169) | | | | | |
| Night of Olay ® | 7.0 | 6.5 | 6.5 | 6.5 | 6.5 |
| Lot 916OH | | | | | |
| Nivea ® Q-10 Cream | 7.0 | 7.0 | 7.0 | 7.0 | 7.0 |
| (Lot 92840851) | | | | | |
| Olay ® Activating | 7.0 | 7.0 | 7.0 | 7.0 | 7.0 |
| Hydration Lotion | | | | | |
| (Lot 901911) | | | | | |
| Estee Lauder ® Resilience | 6.0 | 6.0 | 6.0 | 6.0 | 6.0 |
| (Lot CB9) | | | | | |
| Clinique ® Dramatically | 7.5 | 7.5 | 7.5 | 7.5 | 7.5 |
| Moisturizing Lotion | | | | | |
| (Lot 037) | | | | | |
| Lancôme ® Renergic | 6.0 | 6.0 | 6.0 | 6.0 | 6.0 |
| Antiwrinkle Cream | | | | | |
| (Lot 0257) | | | | | |
| Example 1 | 7.0 | 5.5 | 5.0 | 4.5 | 4.5 |
| Example 2 | 7.0 | 5.8 | 5.0 | 4.5 | 4.5 |
| Example 3 | 7.0 | 5.5 | 5.0 | 4.5 | 4.5 |
| Example 4 | 7.0 | 5.5 | 5.0 | 4.5 | 4.5 |
| Example 5 | 7.0 | 5.5 | 5.0 | 4.5 | 4.5 |

*ColorpHast pH 0-14 pH paper, EM Science, Gibbstown, NJ 08027 was used for this test

### REFERENCES

Berardesca, E., Pirot, F., Singh, M. and Maibach, H. “Differences in stratum corneum pH gradient when comparing white Caucasian and black African-American skin”, Brit. J. Dermatology, 139, 855-857 (1998)
Feingold, K. R. and Elias, P. M. in Dermatology: Clinical & Basic Science Series, eds Marie Loden and Howard I. Maibach, CRC Press, Boca Raton, Fla., 45-58, (2000).
Kligman, A. in Dry Skin and Moisturizers, Eds. Marie Loden and Howard I. Maibach, CRC Press, Boca Raton, Fla., 8 (2000).
Mauro, T. Arch. Dermatol. Res. 290(4): 215-222 (1998).
Nowak, R., “Oil and Water Do Mix After All”, New Scientist, 19, Feb. (2003).
Nowak, R. “Oil and Water Mix for Better Drugs.” New Scientist, 28, pp. March, (2005).

1. A topical composition, comprising an ammonium salt of a weak organic acid in a cosmetically acceptable base formulation wherein the composition releases ammonia when in skin contact to provide a gradual increase in acidity to a skin compatible pH.

2. The composition of claim 1 wherein pH of the base formulation is between pH 6.0 and 7.0 when applied to the skin.

3. The composition of claim 1 wherein the acidity of the composition increases to pH 4.0-4.5 within one to four hours after application to the skin.

4. The composition of claim 1 wherein wherein the acidity of the composition increases to pH 4.0-4.5 within about three hours after application to the skin.

5. The composition of claim 1 wherein the composition further comprises an ingredient selected from the group consisting of tocopheryl acetate, vitamin A palmitate, aloe vera gel, beeswax, sodium hyaluronate, cholesterol, lanolin, fragrance and combinations thereof.

6. The composition of claim 1 wherein the weak organic acid has a pKa between 4.0 to 5.5.

7. The composition of claim 1 wherein the weak organic acid is selected from the group consisting of an alpha hydroxy acid, a beta hydroxy acid, salicylic acid, glycolic acid, lactic acid, malic acid, citric acid and tartaric acid.

8. A packaged formulation for use as a topically applied cosmetic, comprising the composition of claim 1 and directions for use.

9. The composition of claim 1, wherein said composition is a solution, lotion, gel or cream.

10. The composition of claim 1 wherein the cosmetically acceptable base formulation comprises an emulsifier.

11. The composition of claim 10 wherein the emulsifier is selected from the group consisting of cetyl alcohol, ceteth-20, glyceryl stearate, PEG-100 stearate, stearic acid and palmitic acid.

12. A topical cosmetic composition, comprising an ammonium salt of a weak organic acid in a cosmetically acceptable base wherein ammonia is released upon topical application of said composition to provide a gradual decrease in pH to a value determined by the pKa of said ammonium salt and corresponding weak acid formed in the presence of skin moisture.

13. A topical skin delivery system, comprising:
a stabilized formulation having a pH of about 6.5-7.0 prepared from a combination of
a weak organic acid;
a cosmetically acceptable base;
a moisturizer; and
aqueous ammonia;
wherein said formulation gradually increases in acidity when topically applied to the skin to a pH determined at least in part by the pKa of the weak organic and the corresponding ammonium salt.

14. The skin delivery system of claim 13 wherein the weak organic acid provides a pH of about 4.5-5.5 after application to the skin.

15. A packaged formulation for use in topical skin application, comprising a cosmetically acceptable composition comprising a weak organic acid in accordance with claim 1 and instructions for use.

16. The composition of claim 1 further comprising a cosmetically effective agent for enhancing sloughing.

17. The composition of claim 16 wherein the sloughing agent is selected from the group consisting of L-ascorbic acid, alpha hydroxy acids, beta hydroxy acids glycolic acid, phenol, salicylic acid, lactic acid, alpha-keto acids, retinoic acid and resorcinol.

18. The composition of claim 1 further comprising a pharmaceutically effective agent for acne treatment.

19. The composition of claim 18 wherein the pharmaceutically effective agent for acne treatment is a beta-hydroxy acid or salicylic acid in an amount acceptable for acne treatment.

20. A delivery system for providing an on-demand topical skin moisturizing preparation, comprising a means for releasing aliquots of a stabilized pH neutral cosmetic facial composition and a separately compartmentalized cosmetically acceptable acidic composition, wherein mixing of said aliquots results in an acidic mixture directly applicable to the skin.

21. The delivery system of claim 20 wherein the acidic mixture is pH 4.5-5.5.
